(19) United States
(12) Patent Application Publication
(10) Pub. No.: US 2012/0001077 A1
(43) Pub. Date: Jan. 5, 2012

(54) RADIATION TOMOGRAPHY APPARATUS

(76) Inventors: Yoshihiro Inoue, Kyoto-fu (JP); Masaharu Amano, Osaka-fu (JP); Kazumi Tanaka, Shiga-ken (JP); Tetsuro Mizuta, Kyoto-fu (JP); Atsushi Ohtani, Kyoto-fu (JP)

(21) Appl. No.: 13/257,279

(22) PCT Filed: Mar. 25, 2009
(86) PCT No.: PCT/JP2009/001332

§ 371 Date: Sep. 16, 2011

(51) Int. Cl. G01T 1/161; A61B 6/04

(52) U.S. Cl. 250/363.02

(57) ABSTRACT

This invention has one object is to provide radiation tomography apparatus that allows production with low price through suppression in number of radiation detectors to be mounted. One of the detector rings in this invention is a first detector ring having a sufficient internal diameter to introduce shoulders of the subject M, and the other is a second detector ring having a smaller internal diameter than the first detector ring. In so doing, the radiation detectors forming the detector ring may be suppressed in number, which may provide radiation tomography apparatus of low price. Moreover, a smaller diameter of the detector ring may result in improved spatial resolution and detection sensitivity of radiation.

### TECHNICAL FIELD

This invention relates to radiation tomography apparatus that images radiation emitted from a subject. Particularly, this invention relates to radiographic apparatus having a field that is wide enough to image a body portion of the subject at one time.

### BACKGROUND ART

In medical fields, radiation emission computed tomography (ECT: Emission Computed Tomography) apparatus is used that detects an annihilation radiation (for example, gamma rays) pair emitted from radiopharmaceutical that is administered to a subject and is localized to a site of interest for acquiring sectional images of the site of interest in the subject showing radiopharmaceutical distributions. Typical ECT equipment includes, for example, a PET (Positron Emission Tomography) device and an SPECT (Single Photon Emission Computed Tomography) device.

A PET device will be described by way of example. The PET device has a detector ring with block radiation detectors arranged in a ring shape. The detector ring is provided for surrounding a subject, and allows detection of radiation that is transmitted through the subject.

First, description will be given of a configuration of a conventional PET device. As shown in FIG. 9, a conventional PET device 50 includes a gantry 51 with an introducing hole that introduces a subject, a detector ring 53 having block radiation detectors 52 for detecting radiation being arranged inside the gantry 51 as to surround the introducing hole, and a support member 54 provided as to surround the detector ring 53. Each of the radiation detectors 52 has a bleeder unit 55 with a bleeder circuit. The bleeder unit 55 is provided between the support member 54 and the radiation detector 52 for connecting the support member 54 and the radiation detector 52.

The PET device determines annihilation radiation pairs emitted from radiopharmaceutical. Specifically, an annihilation radiation pair emitted from inside of a subject M is a radiation pair having traveling directions opposite by 180 degrees. The detector ring 53 has detecting elements C arranged in a z-direction for detecting an annihilation radiation pair. Accordingly, a position of the annihilation radiation pair relative to the detector ring 53 may be discriminated in the z-direction.

A sectional image of a body portion in the subject M is acquired with use of such radiation tomography apparatus while the subject M is moved relative to the detector ring 53. The subject M is projected from the detector ring 53, and thus a site of interest in the subject M may occasionally be out of the detector ring 53. Accordingly, in the conventional configuration, the sectional image should be taken while a field of view of the detector ring 53 is shifted relative to the subject M.

That is, the detector ring 53 needs to have a hole that is large enough to pass the subject M. Specifically, the detector ring 53 is set to have an internal diameter that is large enough to introduce a shoulder as the widest site in the subject M. Radiation tomography apparatus provided with the detector ring 53 having a small internal diameter has also been invented. However, this apparatus does not aim at imaging of the subject M over a wide range, but is used for head inspection. The radiation tomography apparatus adopting such configuration is described, for example, in Patent Literatures 1 and 2.
[Patent Literature 1]
Japanese Patent Publication (Translation of PCT Application) No. 2004-533607
[Patent Literature 2]
Japanese Utility Model (Registration) Publication No. S63-25395

### DISCLOSURE OF THE INVENTION

### Summary of the Invention

The conventional configuration as above, however, has the following problem. Specifically, adaptation of the conventional configuration directly to radiation tomography apparatus for total body inspection may lead to radiation tomography apparatus of high price. That is, the longer detector ring 53 in the z-direction may cause increase in number of radiation detectors to be mounted. Accordingly, the detector ring 53 greatly increases in manufacturing cost. Recently, radiation tomography apparatus has been developed having the wide detector ring 53 as to cover the entire of the subject. The cost of radiation tomography apparatus is largely influenced by the number of radiation detectors provided therein. Consequently, the detector ring 53 having a smaller internal diameter is preferable.

On the other hand, according to the conventional configuration, the detector ring 53 needs to have an internal diameter that is sufficient to pass the shoulder of the subject M for insertion of the subject M. Accordingly, the detector ring 53 extends in the z-direction without variation in internal diameter for realizing radiation tomography apparatus for total body inspection, which causes increased manufacturing cost.

This invention has been made having regard to the state of the art noted above, and its object is to provide radiation tomography apparatus that allows production with low price through suppression in number of radiation detectors to be mounted.

### Means for Solving the Problem

This invention is constituted as stated below to achieve the above object. That is, radiation tomography apparatus according to this invention includes a first detector ring and a second detector ring each having annularly arranged radiation detectors for detecting radiation from a subject, a bed provided inside the first detector ring and the second detector ring, a bed moving device for moving the bed, and a bed movement control device for controlling the bed moving device. The bed moving device moves the bed, whereby the bed is movable along a connection direction in which the first detector ring and the second detector ring are connected. The bed moves in a direction from the first detector ring toward the second detector ring when the bed is inserted into inside of both the detector rings. The bed moves in a direction from the second detector ring toward the first detector ring when the bed is retracted from inside of both the detector rings. Both the detector rings are arranged in a direction of central axes as to share each central axis. The first detector ring has an internal diameter that is larger than the second detector ring.

### Operation and Effect

This invention includes at least two detector rings for detecting radiation from the subject. One of the detector rings is the first detector ring having a sufficient internal diameter to introduce shoulders of the subject, and the other is the second detector ring having a smaller internal diameter than the first detector ring. The subject has a largest width at the shoulder thereof. Consequently, it is not necessary for the detector ring to have a large internal diameter throughout thereof. The detector ring may have a region with a smaller internal diameter independently of the shoulder of the subject. In so doing, the radiation detectors forming the detector ring may be suppressed in number, which may provide radiation tomography apparatus of low price.

Moreover, a smaller diameter of the detector ring may result in improved spatial resolution and detection sensitivity of radiation. The longer the distance becomes between the radiation detector and a generation source of radiation, the less the dose of radiation reaches the radiation detector. Consequently, in order to improve detection sensitivity, a smaller internal distance between the subject and the radiation detector and a smaller diameter of the detector ring are preferable. Moreover, an annihilation radiation pair is generated through collision of a positron with an electron. Here, kinetic energy of the positron and the electron is conserved in the paired radiation. Consequently, each of the annihilation radiation pair travels in a direction slightly deviating from a straight angle opposite to each other. Accordingly, the incident position in the detector ring deviates from an ideal position. The larger internal diameter the detector ring has, the larger an amount of deviation of the incident position in the detector ring becomes due to deviation in the travel direction of the annihilation radiation pair. Consequently, the radiation tomography apparatus has poor spatial resolution. That is, the detector ring having a smaller internal diameter is preferable for provision of the radiation tomography apparatus of high spatial resolution. According to the configuration of this invention, both two effects mentioned above will be produced.

### Operation and Effect

According to this configuration, the subject may reliably be inserted into inside of the detector rings. Specifically, the bed moves in a direction from the first detector ring toward the second detector ring when the bed is inserted into inside of both the detector rings. That is, the shoulder of the subject is inserted from a side of the first detector ring having a larger internal diameter. Accordingly, the shoulder of the subject does not interfere with the second detector ring even when the bed moves. This applies also to a case where the subject is retracted from the detector rings. That is, in this case the bed moves in a direction from the second detector ring toward the first detector ring. Accordingly, the shoulder of the subject does not interfere with the second detector ring even when the bed moves.

It is more desirable that a coincidence device across detector rings is provided for counting a number of coincidence events as a number of times that two different radiation detectors belonging to the foregoing first detector ring and the second detector ring detect radiation coincidentally.

### Operation and Effect

According to this configuration, coincidence may be performed to an annihilation radiation pair detected across the two detector rings. This invention includes a first coincidence section for performing coincidence to an annihilation radiation pair detected in the first detector ring, and a second coincidence section for performing coincidence to an annihilation radiation pair detected in the second detector ring. This invention further includes the coincidence device across detector rings provided for counting a number of coincidence events as a number of times that two different radiation detectors belonging to the first detector ring and the second detector ring detect radiation coincidentally. Provision of this may realize determination of a single annihilation radiation pair in cooperation with the first detector ring and the second detector ring. Consequently, the amount of data used in the radiation tomography may increase, and thus the radiation tomography apparatus may be provided that allows generation of a clearer sectional image.

Moreover, provided are a bed moving device for moving the foregoing bed, and a bed movement control device for controlling the bed moving device. The bed moving device moves the bed, whereby the bed is movable along a connection direction where the first detector ring and the second detector ring are connected. The bed moves in a direction from the first detector ring toward the second detector ring when the bed is inserted into inside of both the detector rings. The bed moves in a direction from the second detector ring toward the first detector ring when the bed is retracted from inside of both the detector rings. Such configuration is more desirable.

(Deleted).

The foregoing bed has a first portion connected in the connection direction, and a second portion having a narrower width than the first portion in a radial direction of the first detector ring. When the bed is inserted inside of both the rings, the first portion is located inside of the first detector ring, and the second portion inside of the second detector ring. Such configuration is more desirable.

### Operation and Effect

With this configuration, the second detector ring may reliably be reduced in internal diameter. That is, in the foregoing configuration, the bed has a shape along the internal diameter of the detector ring. Specifically, when the bed is inserted inside of both the rings, the first portion is located inside of the first detector ring and the second portion inside of the second detector ring. In addition, when the bed is retracted from inside of both the detector rings, the bed moves in the direction from the second detector ring toward the first detector ring. Consequently, the wide first portion in the bed does not pass the second detector ring, which may avoid interference with each other.

Moreover, the foregoing first portion has an exposure portion at a side end thereof on the second detector ring side where the second portion is not connected. A sensing device is provided for sensing approach of the exposure portion relative to the second detector ring. The bed control device stops movement of the bed in the direction from the first detector ring toward the second detector ring in accordance with sensing of the sensing device. Such configuration is more desirable.

### Operation and Effect

Such configuration may provide radiation tomography apparatus with high safety. The first portion has an exposure portion at a side end thereof on the second detector ring side where the second portion is not connected. The exposure portion may possibly interfere with the second detector ring. According to the foregoing configuration, the sensing device is provided for sensing approach of the exposure portion relative to the second detector ring. Insertion of the bed stops when the exposure portion approaches to the second detector ring to some degree. Therefore, the foregoing configuration may provide radiation tomography apparatus of high safety with no interference of the bed and the second detector ring.

Moreover, it is more desirable that the foregoing bed has a movement restraint device for restraining movement of the bed relative to the subject.

### Operation and Effect

Such configuration may provide radiation tomography apparatus with high safety. Provision of the movement restraint device on the bed may prevent hands of the subject from being inserted between the bed and the second detector ring when the bed is inserted inside of the detector ring. That is because the hands of the subject are held stationary.

Moreover, the foregoing radiation tomography apparatus further includes an image generation device, adjacent to the first detector ring, having (A) a radiation source that allows rotation relative to the bed around the central axis, (B) a radiation detecting device that allows rotation relative to the bed around the central axis, (C) a support device for supporting the radiation source and the radiation detecting device, (D) a rotating device for rotating the support device, and (E) a rotation control device for controlling the rotating device. Such configuration is more desirable.

### Operation and Effect

According to the above configuration, radiation tomography apparatus may be provided that allows acquisition of both images of an internal subject structure and pharmaceutical distribution. In general, a PET device may obtain information on pharmaceutical distribution. However, it may sometimes be necessary to conduct diagnosis referring to the sectional image having internal organs and tissue of the subject falling therein. According to the above configuration, both images of the internal structure of the subject and pharmaceutical distribution may be acquired. Consequently, superimposing both images may realize generation of a composite image suitable for diagnosis. Here, the image generation device and the first detector ring are arranged in the central axis direction of the first detector ring.

Moreover, the first detector ring allows insertion of the shoulder of the subject, and the second detector ring allows insertion of the head or legs of the subject.

### Effect of the Invention

This invention includes at least two detector rings for detecting radiation from the subject. One of the detector rings is the first detector ring having a sufficient internal diameter to introduce the shoulder of the subject, and the other is the second detector ring having a smaller internal diameter than the first detector ring. The detector ring may have a region of a small internal diameter that is independent of the shoulder of the subject. In so doing, the radiation detectors forming the detector ring may be suppressed in number, which may provide radiation tomography apparatus of low price. Moreover, a smaller diameter of the detector ring may result in improved spatial resolution and detection sensitivity of radiation.

### BRIEF DESCRIPTION OF THE DRAWINGS

FIG. 1 is a functional block diagram showing a configuration of radiation tomography apparatus according to Embodiment 1.

FIG. 2 is a view showing a configuration of a detector ring according to Embodiment 1.

FIG. 3 is a perspective view showing a configuration of a radiation detector according to Embodiment 1.

FIG. 4 is a sectional view showing a configuration of a bed according to Embodiment 1.

FIG. 5 is a sectional view showing a configuration of a detector ring according to Embodiment 1.

FIG. 6 conceptually shows each section in detail concerning coincidence counting according to Embodiment 1.

FIG. 7 is a functional block diagram showing a configuration of radiation tomography apparatus according to Embodiment 2.

FIG. 8 is a sectional view showing a configuration of radiation tomography apparatus according to one modification.

FIG. 9 is a plan view showing the configuration of the conventional radiation tomography apparatus.

### DESCRIPTION OF REFERENCES

1 . . . radiation detector
8 . . . CT device (image generation device)
9 . . . radiation tomography apparatus
10 . . . bed
10a . . . first portion
10b . . . second portion
10c . . . exposure portion
10s . . . approaching sensor (sensing device)
10r . . . restraining tool (movement restraint device)
12a . . . first detector ring
12b . . . second detector ring
26c . . . third coincidence section
   (coincidence device across detector rings)
39 . . . rotating mechanism (rotating device)
43 . . . X-ray tube (radiation source)
44 . . . FPD (radiation detecting device)
47 . . . support portion (support device)

### BEST MODE FOR CARRYING OUT THE INVENTION

Next, description will be given of a best mode of radiation tomography apparatus according to Embodiment 1. Gamma rays to be described hereinafter are an example of radiation in Embodiment 1. This invention is adapted for a PET device in Embodiment 1, and is adapted for PET/CT apparatus in Embodiment 2.

### Embodiment 1

<Configuration of Radiation Tomography Apparatus>

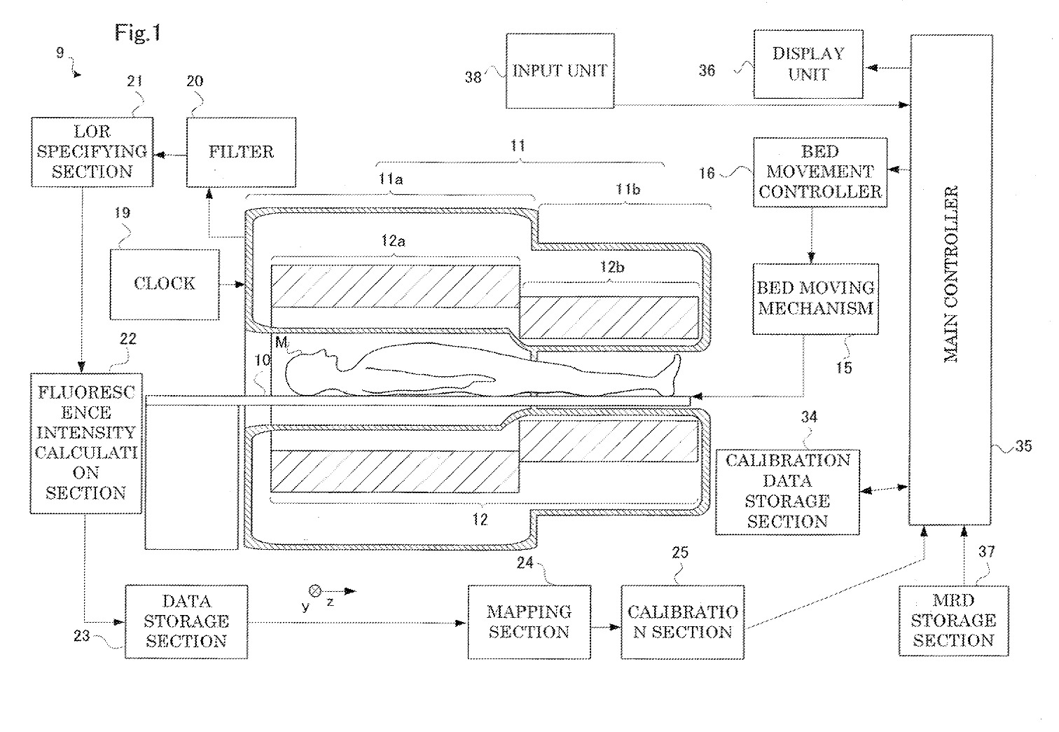

Each embodiment of radiation tomography apparatus according to Embodiment 1 will be described hereinafter with reference to the drawings. FIG. 1 is a functional block diagram showing a configuration of radiation tomography apparatus according to Embodiment 1. As shown in FIG. 1, the radiation tomography apparatus 9 according to Embodiment 1 includes a bed 10 for placing a subject M on the back thereof, and a gantry 11 with a through hole for surrounding the subject M. The bed 10 is provided as to pass through an opening of the gantry 11. The bed 10 freely moves in and out along a direction where the opening of the gantry 11 extends (i.e., a z-direction.) A bed moving mechanism 15 moves the bed 10 as above. A bed movement controller 16 controls the bed moving mechanism 15.

The gantry 11 includes a detector ring 12 inside thereof that detects annihilation gamma-ray pairs from the subject M. The detector ring 12 is tubular and extends in a body axis direction z of the subject M (corresponding to the extension direction of the central axis in this invention.) The detector ring 12 has a length of 1.8 m or more. That is, the detector ring 12 extends as to completely cover a total body of the subject M.

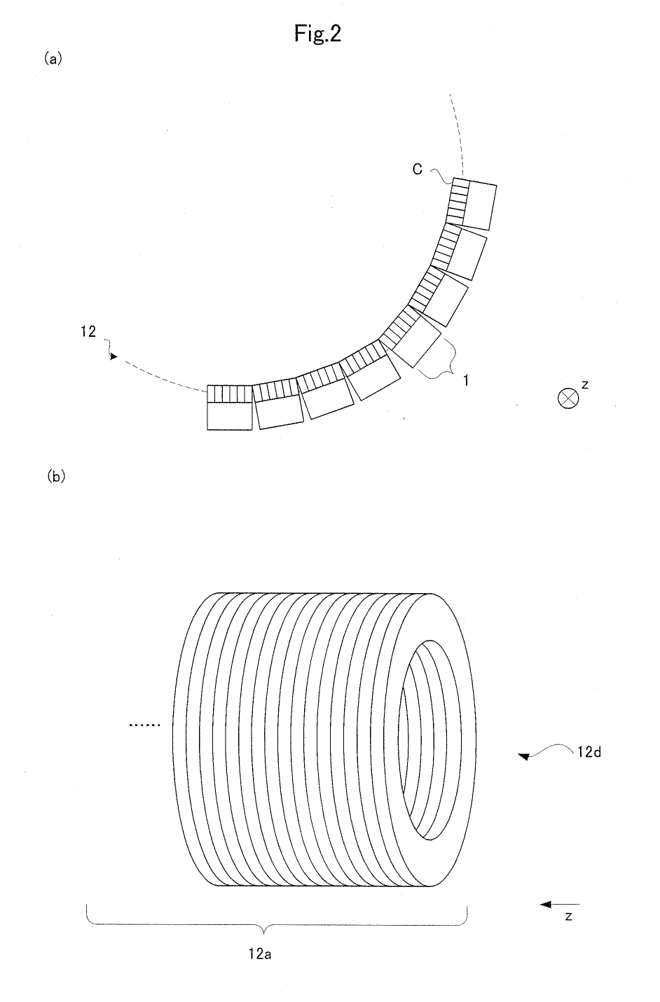

The detector ring 12 according to Embodiment 1 has a first detector ring 12a and a second detector ring 12b arranged (connected to each other) in the z-direction as to share each central axis. As shown in FIG. 2(a), the first detector ring 12a is formed of around one hundred radiation detectors arranged annularly. A through hole 12d is of 100-sided polygon, for instance, seen thereof from the z-direction. FIG. 2(b) is a perspective view of the first detector ring 12a. As above, the radiation detectors 1 are connected in the z-direction to form the first detector ring 12a. Similarly, the radiation detectors 1 are annularly arranged to form the second detector ring 12b. However, the number of radiation detectors 1 forming the second detector ring 12b is fewer than that forming the first detector ring 12a. Here, the first detector ring 12a has an internal diameter of around 650 mm. The second detector ring 12b has an internal diameter of around 300 mm. The gantry 11 is also divided into two parts. The two parts are a first gantry 11a for covering the first detector ring 12a and a second gantry 11b for covering the second detector ring 12b. See FIG. 1.

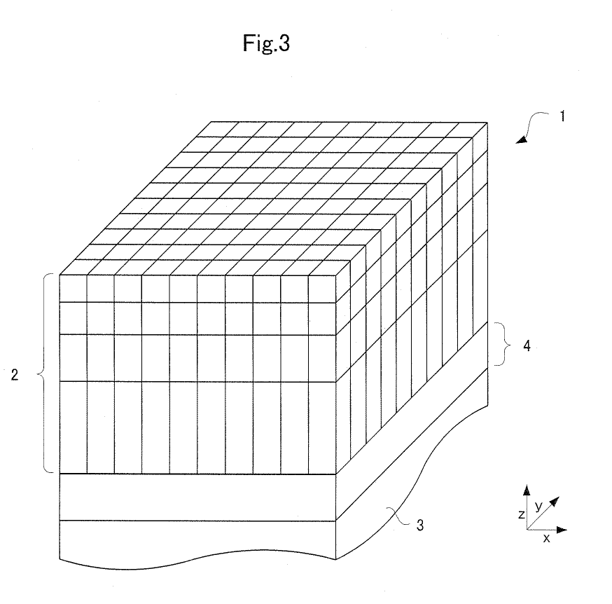

Next, simple description will be given of a configuration of the radiation detector 1. FIG. 3 is a perspective view showing a configuration of the radiation detector according to Embodiment 1. As shown in FIG. 3, the radiation detector 1 includes a scintillator 2 that converts radiation into fluorescence, and a light detector 3 that detects fluorescence. A light guide 4 is provided between the scintillator 2 and the light detector 3 for receiving fluorescence. The configuration of the radiation detector 1 is only one example of embodiments, and is not limited to this.

The scintillator 2 has two or more scintillation counter crystals arranged in a two-dimensional array. Each of the scintillation counter crystals C is composed of Ce-doped Lu_{2(1-X)}Y_{2X}SiO₅ (hereinafter referred to as LYSO.) The light detector 3 allows determination about which scintillation counter crystal emits fluorescence as well as intensity of fluorescence and time when fluorescence is generated.

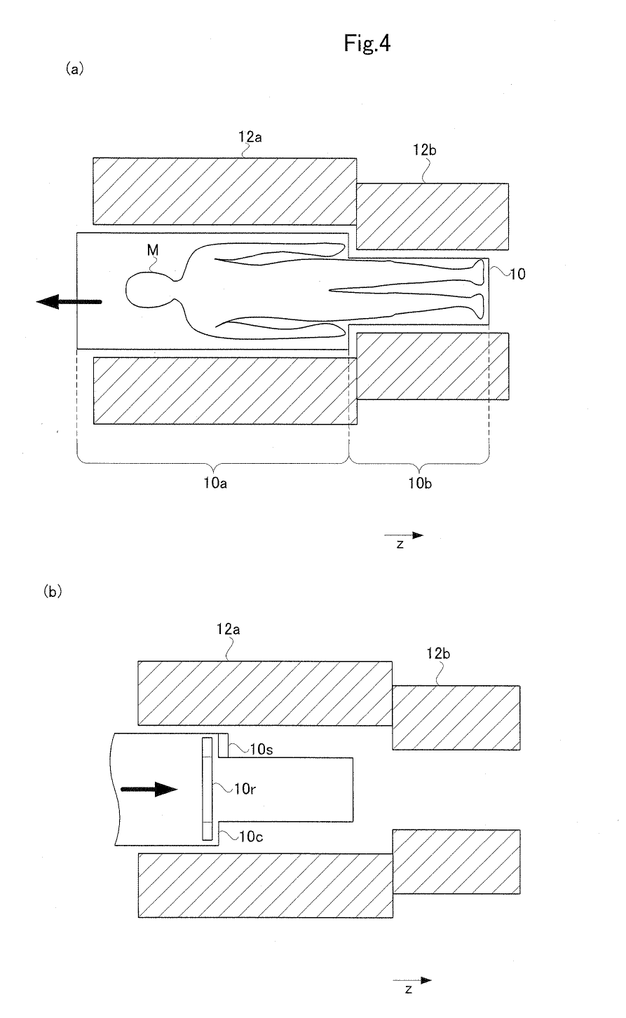

The bed 10 according to Embodiment 1 has a characteristic shape. Specifically, as shown in FIG. 4(a), the bed 10 is formed of the first portion 10a and the second portion 10b connected to each other in the z-direction. The first portion 10a is wide in a radial direction of the first detector ring 12a and the second portion 10b is narrow in the same direction. The first portion 10a supports a head and a body portion of the subject M. The second portion 10b supports legs of the subject M. The shoulder is the widest in the subject M, and thus, the first portion 10a for supporting the shoulder of the subject M should be wide. On the other hand, the second portion 10b has no constrain as above. Accordingly, the second portion 10b may be narrower than the first portion 10a. Here, the radial direction of the first detector ring 12a corresponds to a direction where the bed 10 extends from the radiation detector of the first detector ring 12a toward the central axis (z-axis) of the first detector ring 12a. In other words, it corresponds to a body side direction of the subject M.

The bed moving mechanism 15 is formed of a pulley, a belt, a motor, etc. The bed moving mechanism 15 moves the bed 10 forward/backward in the z-direction in accordance with control of the bed movement control section 16. FIG. 4(a) shows the bed 10 housed inside of the detector ring 12. Here, the first wide portion 10a is located inside of the first detector ring 12a having a large diameter, and the second narrow portion inside of the second detector ring 12b having a small diameter. The bed 10 moves in an arrow direction in FIG. 4(a) for moving the subject M out of the bed 10 from this state. Specifically, the bed 10 moves in a direction from the second detector ring 12b toward the first detector ring 12a when the bed 10 moves out from inside of the detector ring 12.

On the other hand, FIG. 4(b) shows a case where the bed 10 retracted from the detector ring 12 is inserted inside of the detector ring 12. In contrast to this, the bed 10 moves in a direction from the first detector ring 12a toward the second detector ring 12b. Moreover, the first portion 10a and the second portion 10b differ from each other in width. Accordingly, the first portion 10a has an exposure portion 10c at a side end thereof where the second portion 10b is not connected, the exposure portion 10c being not connected to the second portion 10b. The exposure portion 10c is provided with an approaching sensor 10s which output is sent to the bed movement control section 16. The approaching sensor corresponds to the sensing device in this invention.

As the bed 10 is inserted into the detector ring, the exposure portion 10c may interfere with the second detector ring 12b (the second gantry 11b covering thereof, to be exact.) In Embodiment 1, output signals of the approaching sensor 10s are sent to the bed movement controller 16. The bed movement controller 16 controls the bed 10 as to stop when the exposure section 10c approaches the second detector ring 12b to some degree. Accordingly, the bed 10 never interferes with the detector ring 12. Specifically, an infrared sensor may be adopted, for example, as the approaching sensor 10s.

Moreover, the bed 10 has a restraining tool 10r for restraining movement of the bed 10 relative to the subject M. Accordingly, the hands of the subject M may be prevented from being inserted between the bed 10 and the second gantry 11b when the bed 10 is inserted inside of the gantry 11. That is because the hands of the subject M are held stationary. The restraining tool corresponds to the movement restraint device in this invention.

The radiation tomography apparatus 9 according to Embodiment 1 further includes each section for acquiring sectional images of the subject M, as shown in FIG. 1. Specifically, the radiation tomography apparatus 9 includes a filter 20 for extracting effective data from detection data detected in the detector ring 12; a fluorescence intensity calculation section 22 that receives the data determined as the effective data in the filter 20 to obtain fluorescence intensity of an annihilation gamma-rays pair; an LOR specifying section 21 for specifying an incident position of the annihilation gamma-rays pair in the detector ring 12; a data storage section 23 for storing the detection data; a mapping section 24 for generating a sectional image of the subject M; and a calibration section 25 for performing calibration to the sectional image of the subject M. The calibration section 25 removes image artifacts falling in the sectional image with reference to calibration data stored in a calibration data storage section 34. In addition, an MRD storage section 37 stores MRD, mentioned later. An input unit 38 inputs operator's operations. For instance, the input unit 38 receives change of the MRD, for instance.

The radiation tomography apparatus 9 according to Embodiment 1 further includes a main controller 35 for controlling each section en bloc, and a display unit 36 for displaying a radiological image. The main controller 35 is formed of a CPU, and performs execution of various programs to realize the bed movement controller 16, the filter 20, the LOR specifying section 21, the fluorescence intensity calculation section 22, the mapping section 24, and the calibration section 25. The above sections may each be divided into a controller that performs their functions.

<Operation of Radiation Tomography Apparatus>

Next, description will be given of operations of radiation tomography apparatus according to Embodiment 1. Firstly, the subject M is laid on the bed 10 retracted from the detector ring 12 with radiopharmaceutical being administered to the subject M by injection in advance. The bed 10 is introduced inside of the detector rings 12 in accordance with control of the bed movement controller 16. Here, the entire imaging range of the subject M is located inside the detector ring 12. The bed 10 never moves during detection of radiation from the subject M. The positional relationship between the bed 10 and the detector ring 12 is as shown in FIG. 4(a).

An annihilation gamma-rays pair is generated from the subject M, and enters into two different scintillation counter crystals of the detector ring 12. The light detector 3 detects fluorescence generated from the scintillation counter crystals, and outputs detection data. On the other hand, clock data as time information has been sent to the detector ring 12 from the clock 19. For instance, the clock data has such as a serial number in time series order. The clock data is applied (related) to detection data. The clock data to be applied indicates the time when the detector ring 12 detects radiation.

When an annihilation radiation pair enters into the detector ring 12, two pieces of detection data independent of each other are to be outputted from the detector ring 12. Pairing is conducted to the two pieces of detection data, and the detection data is considered derived from a single annihilation radiation pair. Then, detection data to which pairing cannot be conducted is canceled. Such choice of detection data is performed in the filter 20. The filter 20 reads out clock data applied to the detection data, and pass the paired detection data that is simultaneously detected into the subsequent LOR specifying section 21. Here, detection data to which pairing cannot be conducted is canceled.

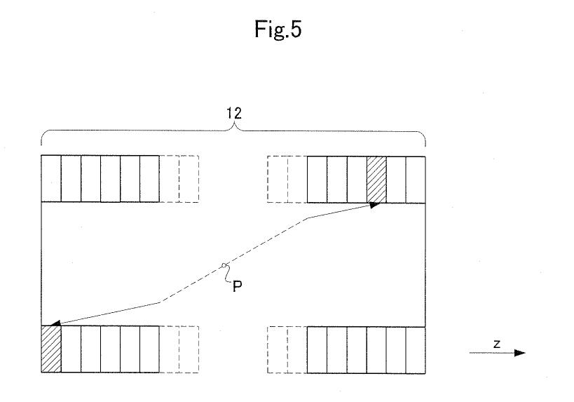

The filter 20 does not pass detection data unconditionally that is detected simultaneously to the LOR specifying section 21. Specifically, the filter 20 passes only detection data suitable for generation of a radiological image into the LOR specifying section 21 with reference to MRD (Maximum ring difference) stored in the MRD storage section 37. That is, as shown in FIG. 5, annihilation gamma rays enter into two scintillation counter crystals far away in the z-direction. Here, annihilation gamma rays are to enter into the scintillation counter crystals further along the z-direction. As show in FIG. 5, it is difficult to detect gamma rays entering into an incident surface of the scintillation counter crystal at a sharp angle, and additionally doses of incident radiation decrease. It is better to dispose of such paired detection data rather than to pass it into the LOR specifying section 21 in terms of reduction in arithmetic load. In Embodiment 1, gamma rays entering at a sharp angle into the incident surface of the scintillation counter crystal are ignored.

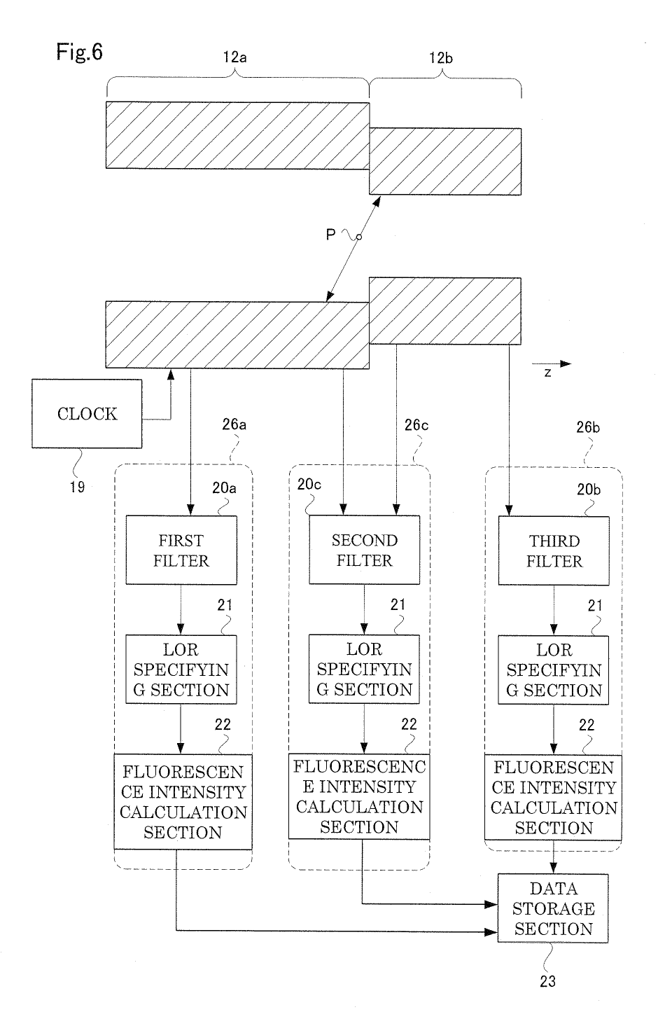

Next, description will be given of a configuration of a coincidence device across detection rings as the characteristic feature in Embodiment 1. FIG. 6 conceptually shows each section in detail concerning coincidence counting according to Embodiment 1. The filter 20 of FIG. 1 includes the first filter 20a, the second filter 20b, and the third filter 20c. The first filter 20a is connected to the first detector ring 12a, and the second filter 20b is connected to the second detector ring 12b. The third filter section 20c is connected to both the first detector ring 12a and the second detector ring 12b. FIG. 6 shows the clock 19 as if it is connected only to the first detector ring 12a. However, the clock 19 is actually connected also to the second detector ring 12b. Here in FIG. 6, the foregoing connection relationship is omitted for brief drawing.

The first filter 20a passes detection data into the LOR specifying section 21 when the first detector ring 12a detects each of annihilation gamma-rays pair. That is, the first filter 20a, the LOR specifying section 21, and the fluorescence intensity calculation section 22 integrally form a first coincidence section 26a for counting a number of coincidence events as a number of times that the annihilation gamma-rays pair is detected in the first detector ring 12a coincidentally. Similarly, the second filter 20b passes detection data to the LOR specifying section 21 when the second detector ring 12b detects each of the annihilation gamma-rays pair. That is, the second filter 20b, the LOR specifying section 21, and the fluorescence intensity calculation section 22 integrally form the second coincidence section 26b.

The third filter 20c passes detection data to the LOR specifying section 21 when the first detector ring 12a detects one of the annihilation radiation pair, and the second detector ring 12b detects the other of the annihilation radiation pair. Specifically, that is a case as shown in FIG. 6 where gamma rays are emitted from a vanishing point P toward both detector rings 12a, 12b. The third filter 20c, the LOR specifying section 21, and the fluorescence intensity calculation section 22 are integrated to count a number of coincidence events as a number of times that two different radiation detectors 1 belonging to the first detector ring 12a and the second detector ring 12b detect radiation coincidentally. That is, the third filter 20c, the LOR specifying section 21, and the fluorescence intensity calculation section 22 form the third coincidence section 26c. Embodiment 1 includes the third coincidence section 26c as above. Accordingly, coincidence may be performed to an annihilation gamma-rays pair across both detector rings 12a, 12b. In addition, clock data correlated with detection data is taken into consideration in determination of coincident property. The third coincidence section corresponds to the coincidence device across detector rings in this invention.

The first filter 20a, the second filter 20b, and the third filter 20c select detection data in consideration of the MRD. Specifically, the filter 20 sends detection data to the LOR specifying section 21 only when two scintillation counter crystals that detect gamma rays coincidentally have a distance in the z-direction of a given value or less indicated with the MRD. The foregoing distance indicated with the MRD is obtained through multiplying a width of the scintillation counter crystal in the z-direction by an integer, and may be set uniquely independent of an arrangement pitch in the z-direction of the radiation detector. The MRD storage section 37 stores the MRD as an integer by which the width of the scintillation counter crystal is to be multiplied in calculation of a given distance.

The LOR specifying section 21 applies radiation intensity to detection data, and specifies an LOR (Line of Response) as a line connecting the two scintillation counter crystals. Specifically, the LOR is a line connecting the scintillation counter crystals different from each other in which gamma rays are considered to enter coincidentally through emitting fluorescence within a given time window. Detection data from the detector ring 12 contains positional information on which scintillation counter crystal emits fluorescence. The LOR specifying section 21 determines an LOR from two pieces of detection data considered to be derived from the annihilation radiation pair. The detection data outputted from the LOR specifying section 21 is stored in the data storage section 23 via the fluorescence intensity calculation section 22. The fluorescence intensity calculation section 22 calculates intensity of gamma rays concerning detection data.

The data storage section 23 stores frequency of detecting the annihilation gamma-ray pair in each LOR. Detection data stored in the data storage section 23 is vector data associated with LORs, fluorescence intensity, and detection time. The mapping section 24 constructs the vector data stored in the data storage section 23 to acquire a sectional image of the subject M. The display unit 36 displays the sectional image acquired in this way. An examination is to be completed.

As above, Embodiment 1 includes at least two detector rings 12 for detecting gamma rays emitted from the subject M. One of the detector rings 12 is the first detector ring 12a having a sufficient internal diameter to introduce the shoulder of the subject M, and the other is the second detector ring 12b having a smaller internal diameter than the first detector ring 12a. The subject M has a largest width at the shoulder thereof. Consequently, it is not necessary for the detector ring 12 to have a large internal diameter throughout thereof. The detector ring 12 may have a region of a smaller internal diameter that is independent of the shoulder of the subject M. In so doing, the radiation detectors 1 forming the detector ring 12 may be suppressed in number, which may provide radiation tomography apparatus 9 of low price. According to this invention, the first detector ring 12a has scintillation counter crystals by approximately 46% of the second detector ring 12b per unit width in the z-direction. Consequently, significant cost reduction may be expected.

Moreover, a smaller diameter of the detector ring 12 may result in improved spatial resolution and detection sensitivity of gamma rays. The longer the distance becomes between the radiation detector 1 and a generation source of gamma rays, the less the dose of gamma rays reaches the radiation detector 1. Consequently, in order to improve detection sensitivity, a smaller internal distance between the subject M and the radiation detector 1 as well as a smaller diameter of the detector ring 1 are preferable. Moreover, an annihilation radiation pair is generated through collision of a positron to an electron. Here, kinetic energy of the positron and the electron is conserved in the annihilation gamma-rays pair. Consequently, each of the annihilation gamma-rays pair travels in a direction slightly deviating from a straight angle opposite to each other. Accordingly, the actual incident position into the detector ring 12 deviates from an ideal position. The larger internal diameter the detector ring 12 has, the larger an amount of deviation from the incident position in the detector ring 12 becomes due to deviation in the travel direction of the annihilation radiation pair. Consequently, the radiation tomography apparatus 9 has poor spatial resolution. That is, the detector ring 12 having a smaller internal diameter is preferable for provision of the radiation tomography apparatus 9 of high spatial resolution. According to Embodiment 1, both two effects mentioned above will be produced.

According to Embodiment 1, coincidence may be performed to an annihilation gamma-rays pair detected across the two detector rings 12. Embodiment 1 includes a first coincidence section 26a for performing coincidence to an annihilation gamma-rays pair detected in the first detector ring 12a, and a second coincidence section 26b for performing coincidence to an annihilation gamma-rays pair detected in the second detector ring 12b. Embodiment 1 further includes a third coincidence device 26c provided for counting a number of coincidence events as a number of times that two different radiation detectors 1 belonging to the first detector ring 12a and the second detector ring 12b detect gamma rays coincidentally. Provision of this configuration may realize determination of a single annihilation gamma-rays pair in cooperation with the first detector ring 12a and the second detector ring 12b. Consequently, the amount of data used in the tomography may increase, and thus the radiation tomography apparatus 9 may be provided that allows generation of a clearer sectional image.

According to Embodiment 1, the subject M may reliably be inserted into inside of the detector ring 12. Specifically, the bed 10 moves in a direction from the first detector ring 12a toward the second detector ring 12b when the bed 10 is inserted into inside of the detector ring 12. That is, the shoulder of the subject M is inserted from a side of the first detector ring 12a having a larger internal diameter. Accordingly, the shoulder of the subject M does not interfere with the second detector ring 12b even when the bed 10 moves. This applies also to a case where the subject M is retracted from the detector ring 12. Specifically, the bed 10 moves in a direction from the first detector ring 12a toward the second detector ring 12b when the bed is retracted from inside of both the detector rings 12a, 12b. Accordingly, the shoulder of the subject M does not interfere with the second detector ring 12b even when the bed 10 moves.

With the configuration of Embodiment 1, the second detector ring 12b may reliably be reduced in internal diameter. That is, in this configuration, the bed 10 has a shape along the inside of the detector ring 12. Specifically, when the bed 10 is inserted inside of the detector ring 12, the first wide portion 10a is located inside of the first detector ring 12a and the second narrow portion 10b inside of the second detector ring 12b. In addition, when the bed 10 is retracted from inside of the detector ring 12, the bed 10 moves in the direction from the second detector ring 12b toward the first detector ring 12a as shown in FIG. 4(a). Consequently, the first wide portion 10a does not pass the second detector ring 12b, which may avoid interference with each other.

Such configuration of Embodiment 1 may provide radiation tomography apparatus 9 with high safety. The first portion 10a has an exposure portion 10c at a side end thereof on the second detector ring 12b side where the second portion 10b is not connected. The exposure portion 10c may possibly interfere with the second detector ring 12b. According to this configuration, the sensing device 10s is provided for sensing approach of the exposure portion 10c relative to the second detector ring 12b. Insertion of the bed 10 stops when the exposure portion 10c approaches to the second detector ring 12b to some degree. Therefore, the foregoing configuration may provide radiation tomography apparatus 9 of high safety with no interference of the bed 10 and the second detector ring 12b.

Such configuration of Embodiment 1 may provide radiation tomography apparatus 9 with high safety. Provision of the movement restraining tool 10r on the bed 10 may prevent hands of the subject M from being inserted between the bed 10 and the second detector ring 12b when the bed 10 is inserted inside of the detector ring 12. That is because the hands of the subject M are held stationary.

### Embodiment 2

Next, description will be given of a PET/CT device according to Embodiment 2. The PET/CT device includes the radiation tomography apparatus (PET device) 9 described in Embodiment 1 and a CT device for generating a sectional image using X-rays, and is medical apparatus that allows generation of a composite image having superimposed sectional images acquired in both devices.

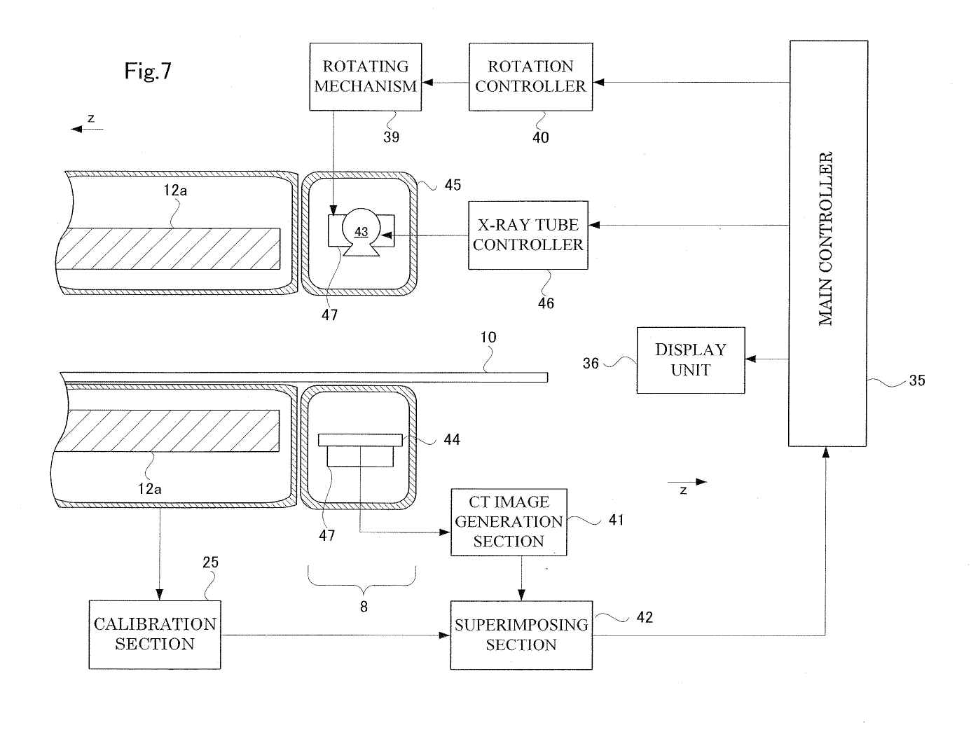

Here, description will be given of a configuration of the PET/CT device according to Embodiment 2. The radiation tomography apparatus (PET device) 9 described in Embodiment 1 may be used for the PET/CT device according to Embodiment 2. Consequently, description will be given of the CT device as a characteristic portion in Embodiment 2. As shown in FIG. 7, the CT device 8 has a gantry 45. The gantry 45 is provided with an opening that extends in the z-direction with a bed 10 inserted therein. Here, the CT device 8 is provided on the first detector ring 12a side of the radiation tomography apparatus 9, and is adjacent to the radiation tomography apparatus 9 in the z-direction.

The gantry 45 has inside thereof an X-ray tube 43 for irradiating a subject with X-rays, an FPD (flat panel detector) 44, and a support portion 47 for supporting the X-ray tube 43 and the FPD 44. The support portion 47 has a ring shape, and freely rotates about the z-axis. A rotating mechanism 39 formed of a power generation device such as a motor and a power transmission device such as a gear performs rotation of the support portion 47. A rotation controller 40 controls the rotating mechanism 39. The X-ray tube corresponds to the radiation source in this invention. The FPD corresponds to the radiation detecting device in this invention. The support portion corresponds to the support device in this invention. The rotating mechanism corresponds to the rotating device in this invention. The rotation controller corresponds to the rotation control device in this invention.

The CT image generation section 41 generates an X-ray sectional image of the subject M in accordance with X-ray detection data outputted from the FPD 44. The superimposing section 42 generates a superimposed image through superimposing the above X-ray sectional image and a PET image showing radiopharmaceutical distribution in the subject that is outputted from the radiation tomography apparatus (PET device) 9.

The CPU 35 performs execution of various programs to realize the mapping section 24, the calibration section 25 according to Embodiment 1 as well as the rotation controller 40, the CT image generation section 41, the superimposing section 42, and the X-ray tube controller 46. The above sections may each be divided into a controller that performs their functions.

Now, description will be given of a method for acquiring an X-ray fluoroscopic image. The X-ray tube 43 and the FPD 44 rotate about the z-axis while a relative position therebetween is maintained. Here, the X-ray tube 43 intermittently irradiates the subject M with X-rays, and the CT image generation section 41 generates an X-ray fluoroscopic image for every irradiation. The two or more X-ray fluoroscopic images are constructed into a single sectional image with use of an existing back projection method, for example, in the CT image generation section 41.

Next, description will be given of a method of generating the composite image. In order to acquire the composite image with the PET/CT device, the site of interest in the subject M is introduced into the CT device to acquire an X-ray sectional image thereof with variation in position of the subject M and the gantry 45. In addition to this, the site of interest in the subject M is introduced into the radiation tomography apparatus (PET device) 9 to acquire a PET image. The superimposing section 42 superimposes both images for completing the composite image. The display unit 36 displays the composite image. Accordingly, radiopharmaceutical distributions and the internal structure of the subject M may be recognized simultaneously, which may result in provision of the sectional image suitable for diagnosis.

According to Embodiment 2, the radiation tomography apparatus 9 may be provided that allows acquisition of both images of pharmaceutical distribution and the internal structure of the subject M. In general, a PET device may obtain information on pharmaceutical distribution. However, it may sometimes be necessary to conduct diagnosis referring to the sectional image having internal organs and tissue of the subject falling therein. According to the above configuration, both images of the internal structure of the subject M and pharmaceutical distribution may be acquired. Consequently, superimposing both images may realize generation of a composite image suitable for diagnosis.

This invention is not limited to the foregoing configuration, but may be modified as follows.

(1) In each of the foregoing embodiments, the scintillation counter crystal is composed of LYSO. Alternatively, the scintillation counter crystal may be composed of another materials, such as GSO (Gd₂SiO₅), may be used in this invention. According to this modification, a method of manufacturing a radiation detector may be provide that allows provision of a radiation detector of low price.

(2) The fluorescence detector in each of the foregoing embodiments is formed of the photomultiplier tube. This invention is not limited to this embodiment. A photodiode, an avalanche photodiode, a semiconductor detector, etc., may be used instead of the photomultiplier tube.

(3) In the foregoing embodiment, the bed is freely movable. This invention is not limited to this. For instance, the bed may be fixed, whereas the gantry 11 may move.

(4) The detector ring in each foregoing embodiment includes the first detector ring 12a and the second detector ring 12b. This invention is not limited to this embodiment. Three or more detector rings having different internal diameters may be provided.

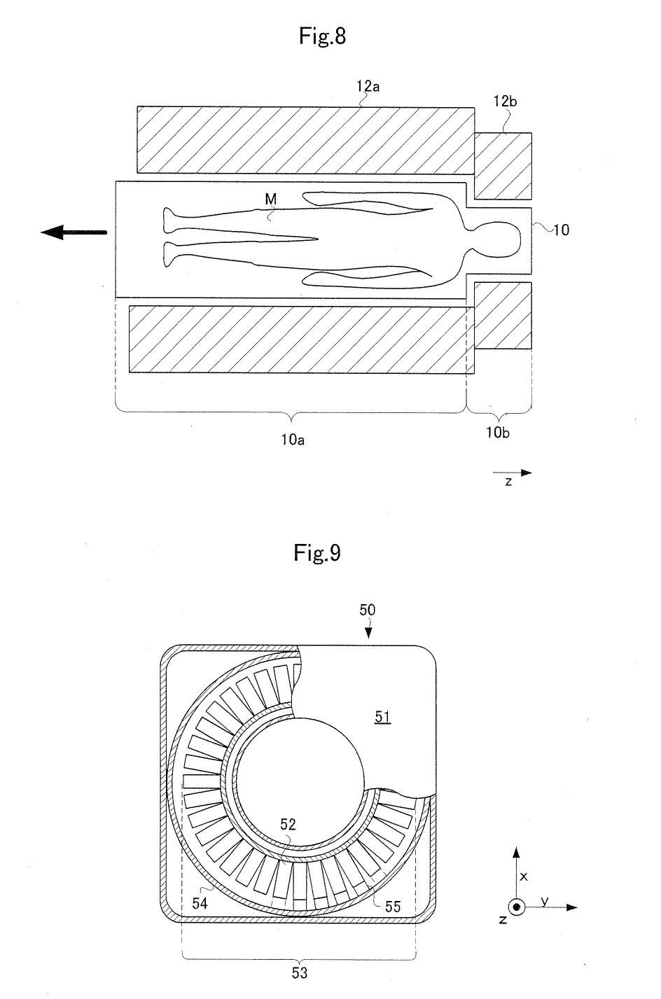

(5) In each foregoing embodiment, the subject M may be inserted from the head thereof, as shown in FIG. 8. The second detector ring 12b in this case has an internal diameter and a length in the z-direction sufficient to cover the head of the subject M. Such configuration may improve spatial resolution at the head. The bed 10 also has a shape along inside of the detector ring 12.

### INDUSTRIAL UTILITY

As described above, this invention is suitable for radiation tomography apparatus for medical uses.

1. Radiation tomography apparatus comprising:
a first detector ring and a second detector ring each having annularly arranged radiation detectors for detecting radiation from a subject;
a bed provided inside the first detector ring and the second detector ring;
a bed moving device for moving the bed; and
a bed movement control device for controlling the bed moving device,
the bed moving device moving the bed, whereby the bed is movable along a connection direction in which the first detector ring and the second detector ring are connected,
the bed moving in a direction from the first detector ring toward the second detector ring when the bed is inserted into inside of both the detector rings,
the bed moving in a direction from the second detector ring toward the first detector ring when the bed is retracted from inside of both the detector rings,
both the detector rings being arranged in a direction of central axes as to share each central axis, and the first detector ring having an internal diameter that is larger than the second detector ring.

2. The radiation tomography apparatus according to claim 1, comprising:
a coincidence device across detector rings for counting a number of coincidence events as a number of times that two different radiation detectors belonging to the foregoing first detector ring and the second detector ring detect radiation coincidentally.

3. (canceled)

4. The radiation tomography apparatus according to claim 1, wherein
the bed has a first portion connected in the connection direction, and a second portion with a narrower width than the first portion in a radial direction of the first detector ring, and
when the bed is inserted inside of both the rings, the first portion is located inside of the first detector ring, and the second portion is located inside of the second detector ring.

5. The radiation tomography apparatus according to claim 4, wherein
the first portion has an exposure portion at a side end thereof on the second detector ring side where the second portion is not connected,
a sensing device is provided for sensing approach of the exposure portion relative to the second detector ring,
the bed movement control device stops movement of the bed in the direction from the first detector ring toward the second detector ring in accordance with sensing of the sensing device.

6. The radiation tomography apparatus according to claim 1, wherein
the bed has a movement restraint device for restraining movement of the bed relative to the subject.

7. The radiation tomography apparatus according to claim 1, further comprising an image generation device, adjacent to the first detector ring, including
(A) a radiation source that allows rotation relative to the bed around the central axis;
(B) a radiation detecting device that allows rotation relative to the bed around the central axis;
(C) a support device for supporting the radiation source and the radiation detecting device;
(D) a rotating device for rotating the support device; and
(E) a rotation control device for controlling the rotating device.

8. The radiation tomography apparatus according to claim 1, wherein
the first detector ring allows insertion of a shoulder of the subject, and
the second detector ring allows insertion of a head or legs of the subject.

9. The radiation tomography apparatus according to claim 2, wherein
the first detector ring allows insertion of a shoulder of the subject, and
the second detector ring allows insertion of a head or legs of the subject.

10. The radiation tomography apparatus according to claim 2, wherein
the bed has a movement restraint device for restraining movement of the bed relative to the subject.

11. The radiation tomography apparatus according to claim 4, wherein
the bed has a movement restraint device for restraining movement of the bed relative to the subject.

12. The radiation tomography apparatus according to claim 5, wherein
the bed has a movement restraint device for restraining movement of the bed relative to the subject.

13. The radiation tomography apparatus according to claim 2, further comprising an image generation device, adjacent to the first detector ring, including
(A) a radiation source that allows rotation relative to the bed around the central axis;
(B) a radiation detecting device that allows rotation relative to the bed around the central axis;
(C) a support device for supporting the radiation source and the radiation detecting device;
(D) a rotating device for rotating the support device; and
(E) a rotation control device for controlling the rotating device.

14. The radiation tomography apparatus according to claim 4, further comprising an image generation device, adjacent to the first detector ring, including
(A) a radiation source that allows rotation relative to the bed around the central axis;
(B) a radiation detecting device that allows rotation relative to the bed around the central axis;
(C) a support device for supporting the radiation source and the radiation detecting device;
(D) a rotating device for rotating the support device; and
(E) a rotation control device for controlling the rotating device.

15. The radiation tomography apparatus according to claim 5, further comprising an image generation device, adjacent to the first detector ring, including
(A) a radiation source that allows rotation relative to the bed around the central axis;
(B) a radiation detecting device that allows rotation relative to the bed around the central axis;
(C) a support device for supporting the radiation source and the radiation detecting device;
(D) a rotating device for rotating the support device; and
(E) a rotation control device for controlling the rotating device.

16. The radiation tomography apparatus according to claim 6, further comprising an image generation device, adjacent to the first detector ring, including
(A) a radiation source that allows rotation relative to the bed around the central axis;
(B) a radiation detecting device that allows rotation relative to the bed around the central axis;
(C) a support device for supporting the radiation source and the radiation detecting device;
(D) a rotating device for rotating the support device; and
(E) a rotation control device for controlling the rotating device.
